(19) United States
(12) Patent Application Publication
(10) Pub. No.: US 2018/0030024 A1
(43) Pub. Date: Feb. 1, 2018

(54) Recovery of tocopherols/tocotrienols, glycerine, sterols and fatty acid esters from vegetable oil distillate and the process thereof

(71) Applicant: DAVID SUE SAN HO, IPOH (MY)

(72) Inventor: DAVID SUE SAN HO, IPOH (MY)

(21) Appl. No.: 15/549,155

(22) PCT Filed: Feb. 4, 2016
(86) PCT No.: PCT/MY2016/050007

§ 371 Date: Aug. 5, 2017

(30) Foreign Application Priority Data
Feb. 6, 2015 (MY) PI2015700389

(51) Int. Cl. C07D 311/72; C11B 3/00

(52) U.S. Cl. CPC C07D 311/72; C11B 3/001

(57) ABSTRACT

A process for recovering tocotrienols/tocopherols from vegetable oil distillate comprising the steps of esterifying the vegetable oil distillate with an alcohol in the presence of an acid catalyst to convert the fatty acids contained therein to produce an esterified mixture comprising fatty acid esters, thereby reducing fatty acid content in the oil to less than 3% by weight; distilling the esterified mixture for separating and removing the fatty acid esters therefrom; transesterifying the distilled mixture with an alcohol in the presence of a basic catalyst for converting glycerides present therein to fatty acid esters and glycerine, forming a transesterified mixture comprising glycerine and fatty acid esters; purifying the transesterified mixture to substantially remove glycerine therefrom; and distilling the purified mixture to substantially remove fatty acid esters, producing a composition comprising a predetermined concentration of tocotrienols/tocopherols.

### FIELD OF INVENTION

The present invention relates to recovery of valuable constituents from vegetable oil distillates from refineries. More particularly, the invention pertains to a multistep process utilizing esterification, distillation and transesterification to recover high quality tocotrienols/tocopherols, sterols and optionally glycerine, as well as free fatty acids in form of ester, from palm oil distillate.

### BACKGROUND OF THE INVENTION

Over the years, crude vegetable oils such as palm, palm kernel, rice bran, soy bean, sunflower, canola, rapeseed, cottonseed, safflower and corn oil have been identified as containing high levels of constituents beneficial to human health. These beneficial constituents may include but not limited to tocopherols, tocotrienols, carotenoids and steroids.

Tocopherols and tocotrienols are valuable constituents of vegetable oils because of their abilities to act as antioxidants and to provide protection against cell damage in the brain, tumors and various types of cancers, as well as to assist in the rehabilitation of damaged cells. Unique molecular structure of the tocotrienols further imparts hypocholesterolemic characteristics, thereby helping to maintain a healthy cardiovascular system. Specifically, tocotrienols can help lower blood cholesterol level through cleansing of the arteries of accumulated cholesterol.

Carotenoids are natural pigments synthesized by plants imparting yellow, orange or red colour. Of all the carotenoids, alpha-carotene, beta-carotene and beta-cryptoxanthin are precursors to vitamin A (or retinol), or interchangeably known as provitamin A, to serve as a source of vitamin A. Other carotenoids such as lutein, lycopene and zeaxanthin cannot be converted to vitamin A but they are still of special interest because they are good antioxidants.

Plant based sterols, on the other hand, are recognized for their abilities to block absorption of cholesterols and reduce blood cholesterol level. Because the plant sterols are nearly identical to the cholesterols, they compete with each other for absorption in the small intestines. However, plant sterols are poorly absorbed by humans and they appear to block the absorption of the dietary cholesterols, thereby reducing the blood cholesterol level, as well as the risk of coronary heart disease. Some research studies have also demonstrated that the plant sterols possess anti-cancer, anti-imflammatory, anti-atherogenic and antioxidant characteristics.

In view of the foregoing, it is desired to extract or recover these beneficial constituents from the crude vegetable oils. Often, during recovery of these constituents, a large fraction of tocotrienols and tocopherols is lost to various distillate streams. These distillate streams typically comprise tocotrienols/tocopherols in an amount of more than 0.10% by weight (or 1000 ppm). However, these streams also comprise high free fatty acid contents which can be more than 10% by weight. Not only free fatty acids, other components such as sterol esters, monoglycerides, diglycerides, triglycerides, glycerine, etc. may also present in the distillate streams. Therefore, in order to obtain a composition concentrated in tocotrienols/tocopherols, it is necessary to remove these undesirable impurities from the oil distillates.

Numerous recovery techniques have been presented in the past, such as solvent extraction, solvent fractionation, ion exchange resin treatment and chromatography method. However, these techniques have several drawbacks, including low yield, substantial degradation of constituents throughout the process, use of carcinogenic organic solvent, etc. Accordingly, there exists a need for an improved method for recovery of tocotrienols/tocopherols.

The present invention provides an improved process for isolating tocotrienols/tocopherols from palm oil distillates, and simultaneously recovering glycerine and free fatty acids in form of esters. In order to further improve quality of the recovered components, acid esterification is utilized as a pretreatment step in the present invention, and then followed by transesterification to remove fatty acids present therein, prior to recovery of tocotrienols/tocopherols.

### SUMMARY OF INVENTION

One of the objects of the invention is to provide a process for isolating tocotrienols, tocopherols and sterols from palm oil distillates, and simultaneously recovering glycerine and free fatty acids in form of esters.

Another object of the invention is to provide a process utilizing acid esterification as a pretreatment step for reducing fatty acid content in the crude oils before being processed further by transesterification, thereby improving quality of the components recovered or isolated from the oils.

Still another object of the invention is to provide a process for isolation and recovery of tocotrienols/tocopherols and optionally other components, such as glycerine and sterols, without being degraded or modified throughout the process.

Yet another object of the invention is to introduce multi-stage distillation as a post-processing step so as to further increase purity of the desirable composition.

At least one of the preceding objects is met, in whole or in part, by the invention, in which one of the embodiments of the invention describes a process for recovering tocotrienols/tocopherols from vegetable oil distillate comprising the steps of esterifying the vegetable oil distillate with an alcohol in the presence of an acid catalyst to convert the fatty acids contained therein to produce an esterified mixture comprising fatty acid esters, thereby reducing fatty acid content in the oil to less than 3% by weight; distilling the esterified mixture for separating and removing the fatty acid esters therefrom; transesterifying the distilled mixture with an alcohol in the presence of a basic catalyst for converting glycerides present therein to fatty acid esters and glycerine, forming a transesterified mixture comprising glycerine and fatty acid esters; purifying the transesterified mixture to substantially remove glycerine therefrom; and distilling the purified mixture to substantially remove fatty acid esters, producing a composition comprising a predetermined concentration of tocotrienols/tocopherols.

In a preferred embodiment of the invention, the vegetable oil distillate is palm fatty acid distillate or any vegetable oil distillate comprising fatty acids of more than 10% by weight of total composition.

According to the previous embodiment, the vegetable oil distillate is subjected to esterification, distillation, transesterification and purification before being subjected to a further distillation. Preferably, the esterification is conducted in presence of an acid catalyst which can be hydrochloric acid, acetic acid, sulphuric acid or citric acid, whilst the transesterification is performed in presence of a basic catalyst such as sodium methoxide or potassium methoxide.

The purifying step mentioned in the foregoing refers to centrifugation or gravitational settling so as to remove glycerine from the transesterified mixture.

In another preferred embodiment of the invention, the process further comprises a step of washing the esterified mixture, or the purified mixture, to remove catalyst, unreacted alcohols and other impurities, followed by heating the washed mixture to reduce moisture content therein to less than 0.07 wt %.

Still another preferred embodiment of the invention is a process for recovering tocotrienols/tocopherols from vegetable oil distillate, further comprising a step of recovering sterols from the composition. The sterols are recovered in crystallised form, as a result of treating the composition with an alcohol and chilling the mixture formed thereof.

Preferably, the alcohol used in esterification, transesterification or sterol recovery step is any one or a combination of methanol, ethanol, propanol and butanol.

One skilled in the art will readily appreciate that the invention is well adapted to carry out the aspects and obtain the ends and advantages mentioned, as well as those inherent therein. The embodiments described herein are not intended as limitations on the scope of the invention.

### DETAILED DESCRIPTION OF THE INVENTION

Hereinafter, the invention shall be described according to the preferred embodiments of the present invention and by referring to the accompanying description and drawings. However, it is to be understood that limiting the description to the preferred embodiments of the invention and to the drawings is merely to facilitate discussion of the present invention and it is envisioned that those skilled in the art may devise various modifications without departing from the scope of the appended claim.

The term “tocotrienol/tocopherol” or “tocopherol/tocotrienol” used herein throughout the description shall refer to any one or a combination of tocotrienols and tocopherols.

The present invention relates to a multistep process involving esterification, distillation, transesterificatiom and further distillation for recovery of tocotrienols/tocopherols from vegetable oil distillates from refineries. Optionally, sterols and glycerine, as well as free fatty acids in form of ester, are recovered from the process provided herein.

It should be appreciated that the vegetable oil distillate referred herein preferably has a composition comprising tocotrienols/tocopherols, sterol esters, fatty acids, monoglycerides, diglycerides and triglycerides, wherein tocotrienols/tocopherols are present in an amount of 0.1 to 5% by weight of total composition, whilst the fatty acids arc present in an amount of more than 10% by weight of total composition.

The process for recovering tocotrienols/tocopherols from vegetable oil distillate, according to its preferred embodiment, comprises the steps of esterifying the vegetable oil distillate with an alcohol in the presence of an acid catalyst to convert the fatty acids contained therein to produce an esterified mixture comprising fatty acid esters, thereby reducing fatty acid content in the oil to less than 3% by weight; distilling the esterified mixture for separating and removing the fatty acid esters therefrom; transesterifying the distilled mixture with an alcohol in the presence of a basic catalyst for converting glycerides present therein to fatty acid esters and glycerine, forming a transesterified mixture comprising glycerine and fatty acid esters; purifying the transesterified mixture to substantially remove glycerine therefrom; and distilling the purified mixture to substantially remove fatty acid esters, producing a composition comprising a predetermined concentration of tocotrienols/tocopherols.

As disclosed in the preferred embodiment of the invention, the vegetable oil distillate comprising tocotrienols/tocopherols is esterified in the presence of an alcohol and an acid catalyst to convert the fatty acids present in the oil to fatty acid alkyl esters, thus forming an esterified mixture comprising glycerides, fatty acid alkyl esters, sterol esters and tocopherols/tocotrienols, thereby reducing the content of the free fatty acids to less than 1%. Preferably, the alcohol used in the esterification is a lower alkyl alcohol such as methanol, ethanol, iso-propanol and butanol, whilst hydrochloric acid, phosphoric acid, citric acid or other suitable acid can be used as the acid catalyst. It should be appreciably noted that the ratio of the acid catalyst to the lower alkyl alcohol in the esterification solution (i.e. the mixture of the acid catalyst and alcohol) may range from 0.005-5:1.

In the preferred embodiment, the acidic esterification is conducted at a temperature of 5 to 90° C. for 0.5 to 6 hours, with or without agitation, but preferably with agitation.

Preferably, after the acid esterification step, the entire esterified mixture is subjected to a heating step, preferably a vacuum evaporation or using the art of equipment of a falling film evaporator, so as to substantially remove the excessive methanol from the mixture. The evaporation may be performed for a period ranging from 1 to 6 hours, at a temperature ranging from 30 to 60° C. The ratio of the methanol being removed to the esterified mixture (i.e. the mixture of the acid catalyst, alcohol and oil distillate) may range from 0.05-0.166:1.

Subsequently, the esterified mixture as obtained is subjected to either direct-current or counter-current water washing, so as to substantially remove the excessive acid catalyst and possibly, unreacted alcohol and other impurities such as sterol esters. Hot or cold water can be used in this step but preferably, water heated to a temperature ranging from 40 to 80° C. is used. In order to ensure optimum removal of the catalyst, alcohol and other impurities from the esterified mixture, the washing step is performed repeatedly until the pH of the esterified mixture achieves a range of 6 to 8.

Resulting from the washing step, the moisture content in the esterified mixture may be increased. It is, therefore, of particular interest to reduce the moisture content therein to less than 0.07% by weight prior to subjecting to further processing. Preferably, the moisture in the washed esterified mixture is reduced by drying, whereby the washed mixture is heated to a temperature of 35 to 80° C. for 0.5 to 10 hours under atmospheric pressure to evaporate the moisture present therein.

Subsequently, the dried esterified mixture is subjected to distillation, in which the distillation is preferably conducted in a pressure lower than atmospheric pressure (also known as “vacuum distillation”). The distillation may involve one or more than one distillation column to produce a first fraction enriched with fatty acid alkyl esters and a second fraction enriched with tocotrienols/tocopherols. In the preferred embodiment of the invention, the distillation is performed in two distillation columns arranged sequentially, both columns operating at a temperature of 120 to 180° C. and a pressure ranging from 0.01 to 1 kPa. Substantial proportion of fatty acid alkyl esters, unreacted fatty acids and other low boiling point substances is separated from the esterified mixture as an overhead stream from the first distillation column. The remaining substances then leave the first distillation column as a residue stream which will be fed into the subsequent column for further separation of fatty acid alkyl esters therefrom. From the subsequent distillation column, fatty acid alkyl esters and possibly, fatly acids are substantially separated to produce a bottom stream, or referred interchangeably to as the “distilled esterified mixture” mentioned in the preceding description, in which the bottom stream comprises tocotrienols/tocopherols in an amount of 1 to 5% by weight of total composition.

In a more preferable embodiment, prior to the distillation, the esterified mixture is subjected to a column dryer to further reduce the moisture content to below 0.01% by weight.

In accordance with the preferred embodiment of the invention, the distilled esterified mixture is subjected to transesterification in the presence of an alcohol and a basic catalyst to convert the glycerides therein to fatty acid esters and glycerine, forming a transesterified mixture comprising glycerine, fatty acid esters, tocotrienols/tocopherols and other impurities such as sterol esters and glycerides. The alcohol used in the transesterification is preferably a lower alkyl alcohol such as methanol, ethanol, iso-propanol and butanol. whilst the basic catalyst can be sodium methoxide or potassium methoxide. Those skilled in the art should appreciate the fact that the ratio of the distilled esterified mixture to the transesterification solution (i.e. the mixture of basic catalyst and alcohol) may range from 0.5-10:1.

Preferably, the transesterification is conducted at a temperature of 5 to 90° C. for 0.5 to 6 hours, with or without agitation, but preferably with agitation. More preferably, prior to transesterification, it is desirable to remove moisture contained therein by heating such that the moisture is evaporated, thereby reducing the moisture content of the first fraction to a level of less than 0.20% by weight.

Further in accordance with the preferred embodiment of the invention, it is desirable to purify the transesterified mixture by separating the glycerine therefrom. It can be achieved by subjecting the transesterified mixture to centrifugation or gravitational settling to produce an ester-rich portion (or referred to as “purified mixture” hereinafter) and a glycerine-rich portion comprising 20 to 40% by weight of glycerine.

The glycerine-rich portion is neutralized using an acid such as hydrochloric acid, acetic acid or sulphuric acid to convert the excessive basic catalyst present therein to salts which can be easily separated from the glycerine, thereby producing 75 to 90% by weight of glycerine.

The purified mixture obtained from the preceding step is preferred to be subjected to direct-current or counter-current water washing for removal of the excessive catalyst and possibly, unreacted alcohol and other impurities such as sterols. Hot or cold water can be used in this step but preferably, water heated to a temperature ranging from 40 to 80° C. is used. Like the preceding washing step, it is preferred to wash the ester-rich portion repeatedly until the pH of the ester-rich portion falls to a range of 6 to 8.

After washing, the moisture content in the purified mixture may be increased and thus, it is desirable to reduce the moisture content therein to less than 0.07% by weight, prior to further processing. It can be achieved by heating the purified mixture to a temperature of 35 to 80° C. for 0.5 to 10 hours, substantially removing the moisture present therein.

Subsequent to the moisture reducing or removal step, the resulting purified mixture is distilled for recovery of tocotrienols/tocopherols. It is worth noting that the distillation herein is similar to that depicted in the foregoing. Specifically, the step for distilling the purified mixture refers to vacuum distillation conducted in two distillation columns arranged in series, the first column operating at a temperature of 120 to 180° C. and at a pressure of 0.01 to 1 kPa, and the subsequent column operating at 120 to 180° C. and 0.01 to 3 kPa. From the first column, fatty acid esters as well as other low boiling point components are primarily recovered as distillate, whilst the components remained unrecovered are discharged as residue stream which is to be fed into the next column for further separation of fatty acid esters therefrom. Substantial portion of fatty acid esters is, again, removed from the next column as distillate. At the same time, a residue stream enriched with tocotrienols/tocopherols is produced, in which the tocotrienols/tocopherols are present in an amount of 5 to 35% by weight. It should be noted that the residue stream is referred interchangeably to as “distilled transesterified mixture”.

In a more preferable embodiment, prior to distillation, the purified mixture is heated, or subjected to a column dryer, to further reduce the moisture content to below 0.01% by weight.

However, those skilled in the art may appreciate the fact that for producing a higher purity stream, or a stream comprising tocotrienols/tocopherols present in a desirable weight percentage, the distilled transesterified mixture can be subjected to another step or more steps of distillation. Alternatively, the distilled transesterified mixture can be recycled to the distillation columns.

Another preferred embodiment of the invention may disclose that the distillate streams obtained in distillation step, comprising predominantly fatty acid esters, can be subjected to further processing or sold directly to fulfill the global market requirement.

Still further in accordance with the preferred embodiment, the distilled transesterified mixture enriched with tocotrienols/tocopherols, is subjected to further processing. Preferably, the distilled transesterified mixture is added with a lower alkyl alcohol, such as any one or a combination of methanol, ethanol, propanol and butanol, to separate sterols in form of crystal. The resulting mixture is continuously agitated and chilled at a temperature ranging from −30 to 0° C. for 12 hours to 3 days, consequently forming crystallised sterols. Subsequently, the mixture is subjected to a solid-liquid separation process for removal of the crystallized sterols. Preferably, the mixture is centrifuged, producing a top layer and a bottom layer.

The bottom layer comprising the sterol crystals are then further processed to evaporate the solvent contained therein through heating or by other drying means.

It should be appreciated that the top layer as obtained from the centrifugation comprises tocotrienols/tocopherols in an amount of 30 to 90% by weight. The top layer may be further heated to evaporate and remove portion of the alcohol present therein, forming a composition having a desirable concentration of tocotrienols/tocopherols. Those skilled in the art may also appreciate that the composition may also contain other compounds such as squalene, sterols and CoQ10.

The disclosure includes as contained in the appended claims, as well as that of the foregoing description. Although this invention has been described in its preferred form with a degree of particularity, it is understood that the disclosure of the preferred form has been made only by way of example and that numerous changes in the details of construction and the combination and arrangements of parts may be resorted to without departing from the scope of the invention.

1-18. (canceled)

19. A process for recovering tocotrienols or tocopherols from vegetable oil distillate, comprising the steps of:
esterifying the vegetable oil distillate with an alcohol in the presence of an acid catalyst to convert fatty acids contained therein to produce an esterified mixture comprising fatty acid esters, thereby reducing a fatty acid content in the oil to less than 3% by weight;
distilling the esterified mixture for separating and removing the fatty acid esters therefrom, in which the step of distilling the esterified mixture is conducted in two distillation columns arranged sequentially, both columns operating at a temperature of 120 to 180° C. and a pressure ranging from 0.01 to 1 kPa, in which a portion of the esterified mixture is removed via an overhead stream from a first distillation column, wherein any remaining substances are fed into a second distillation column for further separation, in which the fatty acids are removed therein to produce a bottom stream comprising tocotrienols or tocopherols in an amount of 1 to 5% by weight of the total composition;
transesterifying the distilled mixture with an alcohol in the presence of a basic catalyst for converting glycerides present therein to fatty acid esters and glycerine, forming a transesterified mixture comprising glycerine and fatty acid esters;
purifying the transesterified mixture to substantially remove glycerine therefrom; and
distilling the purified mixture to substantially remove fatty acid esters, producing a composition comprising a predetermined concentration of tocotrienols or tocopherols, in which the step for distilling the purified mixture is conducted in two distillation columns arranged in series, a first column operating at a temperature of 120 to 180° C. and at a pressure of 0.01 to 1 kPa, and a second column operating at 120 to 180° C. and 0.01 to 3 kPa., in which the first column removes fatty acid esters and the second column further removes unrecovered fatty acid esters thereby producing a residue stream enriched with 5 to 35% by weight of tocotrienols or tocopherols.

20. The process according to claim 19, further comprising the step of:
washing the esterified mixture to remove catalyst, unreacted alcohols and other impurities, followed by heating the washed esterified mixture to reduce moisture content therein to less than 0.07 wt %.

21. The process according to claim 19, further comprising the step of:
washing the purified mixture to remove catalyst, unreacted alcohols and other impurities, followed by heating the washed transesterified mixture to reduce moisture content therein to less than 0.07 wt %.

22. The process according to claim 19, wherein the purifying step is performed to remove glycerine from the transesterified mixture by centrifugation or by gravitational settling.

23. The process according to claim 19, further comprising the step of:
recovering sterols from the composition.

24. The process according to claim 23, wherein the sterols are recovered by treating the composition with an alcohol and chilling the mixture formed thereof, thereby producing crystallised sterols.

25. The process according to claim 20, wherein the washing step is a direct-current or a counter-current water washing step.

26. The process according to claim 21, wherein the washing step is a direct-current or a counter-current water washing step.

27. The process according to claim 20, wherein the washing step is performed using water heated to 40 to 80° C.

28. The process according to claim 21, wherein the washing step is performed using water heated to 40 to 80° C.

29. The process according to claim 20, wherein the washed mixture is heated to a temperature of 35 to 80° C. for 0.5 to 10 hours.

30. The process according to claim 21, wherein the washed mixture is heated to a temperature of 35 to 80° C. for 0.5 to 10 hours.

31. The process according to claim 29, wherein the washed mixture is heated for 0.5 to 10 hours.

32. The process according to claim 30, wherein the washed mixture is heated for 0.5 to 10 hours.

33. The process according to claim 19, further comprising the step of:
heating the esterified mixture, or subjecting the esterified mixture to a dryer, to further reduce the moisture content to below 0.01% by weight.

34. The process according to claim 19, further comprising the step of:
heating the purified mixture, or subjecting the purified mixture to a dryer, to further reduce the moisture content to below 0.01% by weight.

35. The process according to claim 19, wherein the basic catalyst is sodium methoxide or potassium methoxide.

36. The process according to claim 19, wherein the alcohol is any one or a combination of methanol, ethanol, propanol and butanol.

37. The process according to claim 19, wherein the acid is hydrochloric acid, acetic acid, sulphuric acid or citric acid.

38. The process according to claim 19, wherein the vegetable oil distillate is palm fatty acid distillate or any vegetable oil distillate comprising fatty acids of more than 10% by weight of total composition.
